SEQUENCE LISTING (4 sequences)

SEQ ID NO: 1 - RNA, 21 nt, synthetic construct
  source 1..21: mol_type = other RNA
atctataatgatcaggttcat

SEQ ID NO: 2 - RNA, 19 nt, synthetic construct
  source 1..19: mol_type = other RNA
gaacctgatcattatagat

SEQ ID NO: 3 - RNA, 20 nt, synthetic construct
  source 1..20: mol_type = other RNA
ttctgtttgtggaagaactc

SEQ ID NO: 4 - RNA, 20 nt, synthetic construct
  source 1..20: mol_type = other RNA
gagttcttccacaaacagaa

(19) United States
(12) Patent Application Publication
(10) Pub. No.: US 2025/0339540 A1
(43) Pub. Date: Nov. 6, 2025

(54) COMPOSITIONS AND METHODS FOR LOADING EXTRACELLULAR VESICLES

(71) Applicant: Nurexone Biologic Ltd., Haifa (IL)

(72) Inventors: Nisim PERETS, Tel Aviv (IL); Lior SHALTIEL, Modi'in (IL); Lyora AHARONOV, Haifa (IL); Josef MOGRABI, Tel Aviv (IL); Lulu FAHOUM, Nesher (IL)

(21) Appl. No.: 18/869,175

(22) PCT Filed: May 29, 2023
(86) PCT No.: PCT/IL2023/050546

§ 371 Date: Nov. 25, 2024

Related U.S. Application Data
(60) Provisional application No. 63/346,921, filed on May 30, 2022.

(51) Int. Cl. A61K 47/54; A61K 31/7105; A61K 38/16; A61K 47/26; A61K 47/46

(52) U.S. Cl. CPC A61K 47/549; A61K 31/7105; A61K 38/16; A61K 47/26; A61K 47/46

(57) ABSTRACT

The present invention provides extracellular vesicles (EVs) loaded with conjugates of an active agent and a hydrophilic compound such as a carbohydrate, methods for preparation and loading of said EVs, composition comprising the EVs and uses thereof as well as conjugates of active agents and carbohydrates that may be loaded into EVs. In one embodiment, exosomes are loaded with conjugates of a siRNA to glucose.

### FIELD OF THE INVENTION

The present invention is related to compositions and methods for loading extracellular vesicles with active molecules conjugated to hydrophilic compounds such as carbohydrates or derivatives thereof, to the resulting extracellular vesicles and compositions comprising same, wherein the hydrophilic compounds can be biologically active themselves.

### BACKGROUND OF THE INVENTION

Exosomes are membrane-bound extracellular vesicles (EVs) produced in most eukaryotic cells' endosomal compartments. In multicellular organisms, exosomes and other EVs were discovered in biological fluids including blood, urine, and cerebrospinal fluid. Importantly, exosomes were also identified within the tissue matrix, coined Matrix-Bound Nanovesicles (MBV). They are also released in vitro by cultured cells into their growth medium. Since the size of exosomes is limited by that of the parent MVB, exosomes are generally thought to be smaller than most other EVs, from about 30 to 150 nanometers (nm) in diameter: around the same size as many lipoproteins but much smaller than cells. Since exosomes can enter cells naturally and easily, and unload their chemical content inside cells, they can serve as an excellent drug delivery tool for drugs that need to penetrate cells' membrane and accumulate intracellularly. It has been shown that exosomes have many beneficial advantages; they can cross the BBB, have an affinity to inflamed tissues and accumulate in inflamed areas. Exosomes may be an off-the-shelf product that does not require genetic matching. Currently, there are many known methods for loading different compounds into exosomes, such as sonication, electroporation, transfection, incubation, extrusion, saponin-assisted loading, transgenesis, freeze-thaw cycles, thermal shock, pH gradient method, and hypotonic dialysis. In some of these methods, lipophilic compounds such as cholesterol may be used. However, these methods have some disadvantages such as aggregation, disinformation and harm to extracellular vesicles' membrane integrity. Some of the abovementioned methods may affect the targeted cells' ability to engulf the exosomes in a way that the intracellular concentration of the required active ingredient will not be sufficient.

WO2021/030777 relates to EVs (e.g., exosomes) comprising a biologically active molecule covalently linked to the extracellular vesicle via an anchoring moiety, which may be useful as an agent for the prophylaxis or treatment of cancer or other diseases.

EP 3132044 relates to a method of loading exosomes with oligonucleotide cargo, by incubating an oligonucleotide comprising one or more hydrophobic modifications with a population of exosomes for a period of time sufficient to allow loading of the exosomes with the oligonucleotide using genetically engineering of the cells. Such genetic manipulation may change the intrinsic biological characteristic of the cell itself. Therefore, minimal manipulation of the cell is preferable. Further EP3132044 describes exosomes loaded with hydrophobically modified oligonucleotide cargo.

There is still an acute need for additional methods of loading EVs with oligonucleotides and also other different types of desired active ingredients.

### SUMMARY OF THE INVENTION

The present invention discloses compositions and methods for loading extracellular vesicles (EVs) with biologically active molecules. For this, the active molecule is chemically bounded to a non-lipophilic compound that assists in enriching the EVs with the active molecules, and therefore EVs with a high concentration of the active molecules are obtained. It was unexpectedly found that carbohydrates, such as glucose and sucrose, not only enter EVs but may incorporate active agents conjugated with them. It was further found that it is possible to facilitate the loading of EVs with the incorporation of active agents conjugated with glucose by adding insulin to the medium during the loading process.

In some occasions, the non-lipophilic compounds used for loading active agents into EVs are active agents themselves. Thus, the present invention also provides EVs comprising such non-lipophilic active agent compounds. These non-lipophilic compounds may be exogenous compounds and/or present in the EVs at a concentration that does not exist in nature.

According to one aspect, the present invention provides isolated extracellular vesicles comprising at least one exogenous cargo molecule or an exogenous carbohydrate as an active agent, wherein the exogenous cargo molecule comprises an active agent chemically bound to a carbohydrate or derivative thereof. According to one embodiment, the present invention provides isolated extracellular vesicles comprising at least one exogenous carbohydrate as an active agent. According to other embodiments, the present invention provides isolated extracellular vesicles comprising an exogenous cargo molecule comprising an active agent chemically bound to a carbohydrate or derivative thereof. According to some embodiments, the active agent in the cargo molecule is selected from a small molecule, protein, peptide, polypeptide, lipid, and a nucleic acid. According to some embodiments, the active agent carbohydrate is an exogenous carbohydrate. According to some embodiments, the active agent carbohydrate is present in a non-natural concentration. According to some embodiments, the active agent is bound to the carbohydrate or derivative thereof directly or via a linker. According to some embodiments, the linker is a DBCO-C6-acid. According to some embodiments, the active agent is chemically bound to a carbohydrate or derivative thereof via a cleavable linkage. According to some embodiments, the active agent is covalently bound to the carbohydrate. According to some embodiments, the active agent is a nucleic acid. According to some embodiments, the oligonucleotide is selected from RNA, RNAi, siRNA, shRNA, saRNA, miRNA, and miRNA inhibitors. According to some embodiments, the oligonucleotide is siRNA. According to some embodiments, the present invention provides isolated EVs loaded with exogenous cargo molecule comprising siRNA molecule covalently bound to a carbohydrate such as glucose via a linker such as DBCO-C6-acid. According to some embodiments, the present invention provides isolated EVs loaded with exogenous cargo molecule comprising siRNA molecule covalently bound to a carbohydrate such as sucrose via a linker such as DBCO-C6-acid. According to some embodiments, the cargo molecules are present in the EVs in a non-natural concentration, i.e. in a concentration that is not found in nature.

According to another aspect, the present invention provides a method of loading isolated extracellular vesicles (EVs) with exogenous cargo molecules, the method comprises incubating a population of EVs with the cargo molecules comprising an active agent chemically bound to a carbohydrate or derivative thereof. According to some embodiments, the active agent is bound to said carbohydrate or a derivative thereof directly or via a linker. According to some embodiments, the linker is 10-hydroxydecanoic acid. According to some embodiments, the linker is DBCO-C6-acid. According to some embodiments, the active agent is selected from a small molecule, protein, peptide, polypeptide, lipid, and a nucleic acid. According to some embodiments, the active agent carbohydrate is an exogenous carbohydrate and/or present in the EVs in a non-natural concentration.

According to some embodiments, the method further comprises electroporation or the use of a transfection reagent such as a lipid transfection reagent. According to alternative embodiments, the method takes place in the absence of electroporation and in the absence of a transfection reagent. According to some embodiments, wherein the method is performed in the presence of insulin. According to some embodiments, the amount of the loaded exogenous cargo molecule in the resulting EVs is at least 20% higher than in EVs loaded in the absence of insulin.

According to any one of the above aspects and embodiments, the EVs are exosomes. According to some embodiments, the EVs, such as exosomes, are derived from adherent cells expressing mesenchymal markers. According to some embodiments, the adherent cells expressing mesenchymal markers are mesenchymal stem cells (MSC). According to some embodiments, the mesenchymal stem cells are human bone marrow mesenchymal stem cells.

According to some embodiments, the present invention provides isolated EVs obtainable or obtained by the methods described herein.

According to yet another aspect, the provided herein is a pharmaceutical composition comprising a population of the isolated EVs of the present invention, and pharmaceutically acceptable excipients.

According to still another aspect, provided herein is a method of delivering an active agent comprising exposing a mammal, organ, tissue, or a target cell to the isolated EVs of the present invention.

According to another aspect, the present invention provides a method of treating or preventing a disease, medical condition or disorder treatable by the active agent loaded into the EVs, the method comprises administering to a subject in need thereof a therapeutically effective amount of the EVs as described herein.

According to yet another aspect, the present invention provides an exogenous conjugate molecule comprising a nucleic acid chemically bound to a carbohydrate or derivative thereof. According to some embodiments, the nucleic acid is an oligonucleotide.

According to some embodiments, the oligonucleotide is selected from RNA, RNAi, siRNA, shRNA, saRNA, miRNA, and miRNA inhibitor. According to some embodiments, the nucleic acid is bound to a carbohydrate or derivative thereof directly or via a linker. According to some embodiments, the bond or the linker is a cleavable bond or linker. According to some embodiments, the present invention provides siRNA conjugated with glucose. According to some embodiments, the present invention provides siRNA conjugated with sucrose.

According to any one of the above aspects and embodiments, the carbohydrate is selected from a monosaccharide, disaccharide, trisaccharide, tetrasaccharide and oligosaccharide and wherein the carbohydrate derivative is selected from a saccharide linked to an amino acid, polyphenol, or lipid. According to some embodiments, the monosaccharide is selected from glucose, ribose, mannose, arabinose, galactose and xylose; the disaccharide is selected from sucrose, lactose and maltose; the trisaccharide is selected from maltotriose and raffinose; a saccharide linked to an amino acid is D-ribose-L-cysteine; a saccharide linked with a polyphenol is selected from (-)-epigallocatechin gallate 3′-O-α-D-glucoside, isoquercitrin, baicalin and puerarin; and a saccharide linked with a lipid is a cerebroside, such as glucocerebroside.

### BRIEF DESCRIPTION OF THE DRAWINGS

FIG. 1 shows the integrity of exosomes

FIG. 2 shows the absorption curve of sucrose adsorption to exosomes. FIG. 3 shows HPLC analysis of PTEN siRNA 1962 conjugated to D-glucose. FIG. 4A shows a schematic representation of siRNA bound to a carbohydrate via a linker. FIG. 4B shows a schematic representation of siRNA bound to a glucose via linker.

FIG. 5 shows that conjugation of siRNA with glucose does not affect the siRNA's activity: NUR001—anti-PTEN siRNA; competitor loading—the conjugate of the siRNA with cholesterol; NurExo-Load-the conjugate of the siRNA with glucose as described in Example 3.

FIG. 6 shows the loading efficacy of siRNA conjugated with glucose (NurExo-Load) or cholesterol (competitor loading).

FIG. 7 shows the cellular uptake of loaded extracellular vesicles (EVs) in human neural progenitor cells. ReNcell VM cells show a similar uptake of DID-labeled EVs (in violet) loaded with siRNA against PTEN (in green) using either glucose (FIG. 7B) or cholesterol (FIG. 7A), as observed by super-resolution microscopy.

FIG. 8 shows the co-localization analysis of EVs and glucose fluorescent signals.

FIG. 9 shows motor rehabilitation assessed by the evaluation of the BBB score.

FIG. 10 shows improvement of the sensory recovery evaluated with Von Frey filaments.

FIG. 11 shows a decrease in self-eating tendency.

FIG. 12 shows a recovery of tail and paw pinch reflexes.

### DETAILED DESCRIPTION OF THE INVENTION

Unless otherwise defined, all technical and/or scientific terms used herein have the same meaning as commonly understood by one of ordinary skill in the art to which the invention pertains. In case of conflict, the patent specification, including definitions, will control.

The present invention provides extracellular vesicles (EVs) loaded with a cargo molecule comprising an active agent chemically bound to at least one carbohydrate or a derivative thereof. Also, the present invention provides extracellular vesicles (EVs) loaded with an exogenous carbohydrate as an active agent.

The present invention also provides novel methods for loading EVs cargo molecules including hydrophilic compounds bound to an active agent. Non-limiting examples of the hydrophilic compounds are carbohydrates or conjugates thereof. The method of loading extracellular vesicles (EVs) with cargo molecules comprises incubating a population of EVs with the cargo molecules, i.e. active agent chemically bound with at least one carbohydrate or derivatives thereof.

As shown in the examples, carbohydrates provide a similar capacity to load active agents conjugated to them into EVs as cholesterol, which is widely used for this purpose.

Using carbohydrates, and especially sucrose and glucose for incorporation of active agents into EVs also enriches the content of glucose in the EVs. This may be used for example for providing/supplementing cells, especially cells in damaged (e.g. inflamed) tissue. Using sucrose provides cells with even more energy. In addition, using the saccharide for loading EVs does not affect the properties of the EVs' bi-layer contrary to cholesterol, that may increase the rigidity of the membrane. Moreover, this is correct for saccharides whose uptake into EVs is performed via channels. Even more, using saccharides and in particular glucose, it is possible to control the uptake process of the active agent conjugated with saccharide, for example by using insulin.

According to one aspect, the present invention provides isolated extracellular vesicles

(EVs) comprising at least one exogenous carbohydrate as an active agent.

According to another aspect, the present invention provides isolated extracellular vesicles (EVs) comprising a cargo molecule, wherein the cargo molecule comprises an active agent chemically bound to a carbohydrate or derivative thereof. In some embodiments, the cargo molecule is referred to as a conjugate.

According to some embodiments, the cargo molecule is loaded onto the EVs. Thus, according to some embodiments, the present invention provides isolated extracellular vesicles comprising at least one cargo molecule, wherein the cargo molecule comprises an active agent chemically bound to a carbohydrate or derivative thereof. According to any one of the embodiments of the invention, the cargo molecule is an exogenous molecule.

Therefore, according to some embodiments, the present invention provides isolated extracellular vesicles comprising an exogenous cargo molecule, wherein the exogenous cargo molecule comprises an active agent chemically bound to at least one carbohydrate or derivative thereof.

According to some embodiments, the active agent is selected from a small molecule, protein, peptide, polypeptide, lipid, a carbohydrate and nucleic acid. According to some embodiments, the active agent is selected from a small molecule, protein, peptide, polypeptide, lipid and nucleic acid. According to some embodiments, the active agent is selected from a small molecule, lipid, and nucleic acid.

According to some embodiments, the active agent carbohydrate is an exogenic carbohydrate.

The below-provided terms, definitions and embodiments refer to, apply and are encompassed by any one of the aspects of the present invention.

The term “exogenous” refers to a molecule or substance (e.g., a compound, nucleic acid or protein) that is not normally present in a cell or vesicle, and originates from outside and is introduced into the recipient cell or vesicle.

The terms “extracellular vesicles” and “EVs” are used herein interchangeably and refer to cell-derived vesicles comprising a membrane that encloses an internal space. Generally, EVs range in diameter from 30 nm to 1500 nm, more frequently from 40 to 1200 nm, and may comprise various cargo molecules either within the internal space, displayed on the external surface of the extracellular vesicle, and/or spanning the membrane. Said cargo molecules may comprise nucleic acids, proteins, carbohydrates, lipids, small molecules, and/or combinations thereof. The term EVs comprises also the terms “exosome” and “microvesicles”. The terms “exosomes” and “nanovesicle” are used herein interchangeably and refer to EVs having the size of between 30 to 150 nm in diameter. In some embodiments, the term exosomes refer to EVs having the size of between 30 to 100 nm in diameter. The term “microvesicles” as used herein refers to EVs having the size of between 100 to 1000 nm in diameter. Generally, the EVs may comprise at least a part of the molecular contents of the cells from which they are originated, e.g. lipids, fatty acids, polypeptides, polynucleotides, proteins, and/or saccharides.

The EVs are derived from cells. The terms “derived from” and “originated from” are used herein interchangeably and refer to vesicles that are produced within, by, or from, a specified cell, cell type, or any population of cells. As used herein, the terms “parent cell”, “producer cell” and “original cell” include any cell from which the extracellular vesicle is derived. For example, a “parent cell” or “producer cell” includes a cell that serves as a source for the extracellular vesicle. According to some embodiments, the cells are eukaryotic cells.

The extracellular vesicles (EVs) may be derived from biological cells by any of several means, for example by secretion, budding or dispersal from the biological cells. The EVs may be isolatable from a mesenchymal stem cell (MSC), neural crest cell (NCC), mesenchymal stem cell conditioned medium (MSC-CM) or neural crest cell conditioned medium. For example, the EVs may be produced, exuded, emitted or shed from biological cells. Where the biological cell is in cell culture, the EVs may be secreted into the cell culture medium.

Examples of biological cells from which the EVs may be derived include, adherent cells which express mesenchymal markers such as mesenchymal stem cells, oral mucosa stem cells or olfactory ensheathing cells, astrocytes, and neural crest cells. Thus, according to some embodiments, the EVs are derived from adherent cells expressing mesenchymal markers. According to one embodiment, the adherent cells expressing mesenchymal markers are selected from mesenchymal stem cells (MSC), oral mucosa stem cells and olfactory ensheathing cells. According to one embodiment, the cells are mesenchymal stem cells (MSC).

The term “mesenchymal stem cells” refers to multipotent stromal cells that can differentiate into a variety of cell types, as well known in the art, including to: osteoblasts, chondrocytes, myocytes, adipocytes, osteocytes, fibroblasts, and astrocytes.

In their pluripotent state, mesenchymal stem cells typically express the following markers: CD105, CD166, CD29, CD90, and CD73, and do not express CD34, CD45 and CD133.

Mesenchymal stem cells may be isolated from a variety of tissues including but not limited to bone marrow, adipose tissue, dental pulp, oral mucosa, peripheral blood and amniotic fluid. According to some embodiments of the current invention, the mesenchymal stem cells are isolated from bone marrow. According to some embodiments, the mesenchymal stem cells are originated from a site selected from bone marrow, adipose tissue, umbilical cord, dental pulp, oral mucosa, peripheral blood and amniotic fluid.

According to some embodiments, the EVs are derived from bone marrow-originated MSC. According to other embodiments, the EVs are derived from the adipose tissue originated MSC. According to some such embodiments, the EVs are selected from exosomes, microvesicles and a combination thereof. According to some embodiments, the cells express CD105, CD166, CD29, CD90, and CD73 markers. According to a further embodiment, the cells express CD105, CD166, CD29, CD90, and CD73, and do not express CD34, CD45 and CD133. According to some embodiments, the cells are selected from dental pulp stem cells (DPSCs), exfoliated deciduous teeth stem cells (SHED), periodontal ligament stem cells (PDLSCs), apical papilla stem cells (SCAP) and dental follicle progenitor cells (DFPCs).

According to some such embodiments, the EVs comprise or express at least a fraction of the markers expressed by the cell from which EVs are derived.

The EVs may comprise one or more proteins, oligonucleotides or polynucleotides secreted by a particular cell type, e.g. mesenchymal stem cell or neural crest cell. The EVs may comprise one or more proteins or polynucleotides present in mesenchymal stem cell conditioned medium (MSC-CM). In a particular embodiment, the EVs may comprise miRNAs which are derived from MSCs or neural crest cells. For example, the EVs may comprise 10% or more, 20% or more, 30% or more, 40% or more, 50% or more, 60% or more or 70% or more of these proteins and/or polynucleotides. The EVs may comprise substantially about 75% of these proteins and/or polynucleotides. The proteins may be defined by reference to a list of proteins or gene products of a list of genes.

The EVs may have at least one property of a mesenchymal stem cell. The EVs may have a biological property or a biological activity. The EVs may have any of the biological activities of an MSC. The particle may for example have a therapeutic or restorative activity of an MSC.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Mesenchymal stem cell cultures may be generated by diluting BM aspirates (usually 20 ml) with equal volumes of Hank's balanced salt solution (HBSS; GIBCO Laboratories, Grand Island, NY, USA) and layering the diluted cells over about 10 ml of a Ficoll column (Ficoll-Paque; Pharmacia, Piscataway, NJ, USA). Following 30 minutes of centrifugation at 2,500×g, the mononuclear cell layer is removed from the interface and suspended in HBSS. Cells are then centrifuged at 1,500×g for 15 minutes and resuspended in a complete medium

(MEM, a medium without deoxyribonucleotides or ribonucleotides; GIBCO); 20% fetal calf serum (FCS) derived from a lot selected for the rapid growth of MSCs (Atlanta Biologicals, Norcross, GA); 100 units/ml penicillin (GIBCO), 100 g/ml streptomycin (GIBCO); and 2 mM L-glutamine (GIBCO). Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold (Colter DC., et al., Proc Natl Acad Sci USA. 97:3213-3218, 2000).

MSC cultures utilized by some embodiments of the invention include three groups of cells which are defined by their morphological features: small and agranular cells (referred to as RS-1, hereinbelow), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

The EVs may be produced or isolated in a number of ways. Such a method may comprise isolating the EVs from mesenchymal stem cells (MSC) or from neural crest cells (NCC).

According to some embodiments, the EVs of the present invention are isolated EVs.

The EVs of the present invention are mostly spherical and the terms “size”, “particle size”, “average particle size” and “particle diameter size” used herein interchangeably refer to the diameter of the EVs or to the longer diameter of the extracellular vesicles. Any known method for measurement of particle size may be used to determine the size of the EVs of the present invention. A non-limiting example is nanoparticle-tracking analysis (NTA).

According to some embodiments, the EVs are exosomes. According to some embodiments, the EVs are microvesicles. According to a further embodiment, the EVs are a combination of small and large vesicles.

According to any one of the above embodiments, the EVs are isolated. The EVs may be isolated from the cells by standard isolation and washing protocol by differential centrifugation, size exclusion or any other method for particles isolation protocol from the medium.

The terms “purify,” “purified,” “purifying”, “isolate”, “isolated,” and “isolating” are used herein interchangeably and refer to the state of a population (e.g., a plurality of known or unknown amount and/or concentration) of extracellular vesicles, that have undergone one or more processes of purification/isolation, e.g., a selection of the desired extracellular vesicles, or alternatively a removal or reduction of residual biological products and/or removal of undesirable extracellular vesicles, e.g. removing EVs having a particular size. According to one embodiment, the ratio of EVs number to residual parent cells number is at least 2, 3, 4, 5, 6, 8 or 10 times higher, or in certain advantageous embodiments at least 50, 100, 1000, or 2000 times higher than in the initial material. In some advantageous embodiments, the term “isolated” has the meaning of substantially cell-free or cell-free and may be substituted by it. According to some embodiments, the extracellular vesicles, e.g. exosomes, are derived from adherent cells expressing mesenchymal markers. According to some embodiments, the adherent cells expressing mesenchymal markers are mesenchymal stem cells (MSC).

The terms “cargo” and “payload” are used herein interchangeably and include but are not limited to the group consisting of therapeutic agents, diagnostic probes, peptides, nucleic acids, oligonucleotides, antisense oligonucleotides, plasmids, proteins, small molecules, radioactive materials and conjugates therefore, in particular with carbohydrate, loaded and present within the EVs or on their membranes. The terms “cargo” and “conjugate” may be used interchangeably in some embodiments. The term “conjugate” refers to the association between molecules. The association can be direct or indirect. For example, a conjugate between a nucleic acid and a carbohydrate can be direct, e.g., by a covalent bond, or indirect, e.g., by a non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). The terms also refer to an active agent chemically bound to at least one carbohydrate or a derivative thereof. The term “chemically bound” refers both to covalent and non-covalent bonds. According to some embodiments, the active agent is selected from a small molecule, protein, peptide, lipid polypeptide, carbohydrate and nucleic acid. According to some embodiments, the active agent is a small molecule. According to some embodiments, the active agent is a protein. According to some embodiments, the active agent is a peptide. According to some embodiments, the active agent is a lipid polypeptide. According to some embodiments, the active agent is a nucleic acid. According to some embodiments, the active agent is covalently bound to a carbohydrate. According to other embodiments, the active agent is bound to a carbohydrate via non-covalent bonds.

According to some embodiments, the active agent may be a pharmacological agent such as small molecules, nucleic acids, peptides, carbohydrate and proteins. According to some embodiments, the active agent carbohydrate is an exogenous carbohydrate.

The term “active agent”, “pharmacological agents” and “active moiety” are used herein interchangeable and refer to an agent that has biological activity, pharmacologic effects and/or therapeutic utility.

According to some embodiments, pharmacological agent/active agent is an anticancer agent, a cytostatic agent, a DNA or RNA intercalator, a splicing modulator, a tyrosine kinase inhibitor, a statin, an NSAID, an antibiotic, an antifungal agent, an antibacterial agent, an anti-inflammatory agent, an anti-fibrotic, an antihypertensive, an analgesic, an antipyretic, appetite suppressant and weight loss inducer, sedative, sleeping aid, anticonvulsant, hormone, neurotransmitter, an aromatase inhibitor, an esterase inhibitor, an anticholinergic, an SSRI, a BKT inhibitor, a PPAR agonist, a HER inhibitor, an AKT inhibitor, a BCR-ABL inhibitor, a signal transduction inhibitor, an angiogenesis inhibitor, a synthase inhibitor, an ALK inhibitor, a BRAF inhibitor, a MEK inhibitor, a PI3K inhibitor, a neprilysin inhibitor, a beta2-agonist, a CRTH2 antagonist, an FXR agonist, a BACE inhibitor, a sphingosine-1-phosphate receptor modulator, a MAPK inhibitor, an Hedgehog signaling inhibitor, an MDM2 antagonist, an LSD1 inhibitor, a lactamase inhibitor, a TLR agonist, a TLR antagonist, an IDO inhibitor, an ERK inhibitor, a Chk1 inhibitor, a nucleic acid-based agent such as an oligonucleotide, siRNA, shRNA, antisense oligonucleotide, splice-switching oligonucleotide, mRNA, a peptide, a natural product, a polypeptide, a carbohydrate and any combination thereof.

According to any one of the above embodiments and aspects, the carbohydrate is selected from a monosaccharide, disaccharide, trisaccharide, tetrasaccharide, and oligosaccharide.

According to some embodiments, the carbohydrate is a monosaccharide. According to some embodiments, the monosaccharide is selected from glucose, fructose ribose, arabinose, galactose, mannose and xylose. According to some embodiments, the monosaccharide is glucose. According to some embodiments, the monosaccharide is fructose. According to some embodiments, the monosaccharide is arabinose.

According to some embodiments, the carbohydrate is a disaccharide. According to some embodiments, the disaccharide is selected from sucrose, lactose and maltose. According to some embodiments, the disaccharide is sucrose.

According to some embodiments, the carbohydrate is a trisaccharide. According to some embodiments, the trisaccharide is selected from maltotriose and raffinose.

According to some embodiments, the carbohydrate is a tetrasaccharide.

According to some embodiments, the carbohydrate is an oligosaccharide.

According to some embodiments, the carbohydrate derivative is selected from a conjugate of a saccharide with an amino acid, a polyphenol, or lipid.

According to some embodiments, the carbohydrate derivative is a conjugate of a carbohydrate with an amino acid. According to some embodiments, the carbohydrate derivative comprises a carbohydrate linked with an amino acid. According to some embodiments, the saccharide is selected from a monosaccharide, disaccharide, trisaccharide, tetrasaccharide, and oligosaccharide. According to some embodiments, the saccharide is selected from-glucose, -mannose, ribose, -arabinose, -galactose, sucrose and maltotriose. The term “amino acid” as used herein refers to an organic compound comprising both amine and carboxylic acid functional groups, which may be either a natural or non-natural amino acid. The twenty-two natural amino acids are aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl). According to some embodiments, the amino acid is L-cysteine. According to some embodiments, the carbohydrate derivative is D-ribose-L-cysteine.

According to some embodiments, the carbohydrate derivative is a conjugate of a carbohydrate with a polyphenol. According to some embodiments, the carbohydrate derivative comprises a carbohydrate linked with a polyphenol. According to some embodiments, the saccharide is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to some embodiments, the polyphenol is selected from flavonoids and isoflavonoids. According to some embodiments, the conjugate of saccharide with a polyphenol is selected from (-)-epigallocatechin gallate 3′-O-α-D-glucoside, isoquercitrin, baicalin and puerarin.

The compound (-)-epigallocatechin gallate 3′-O-α-D-glucoside has a structure of formula I.

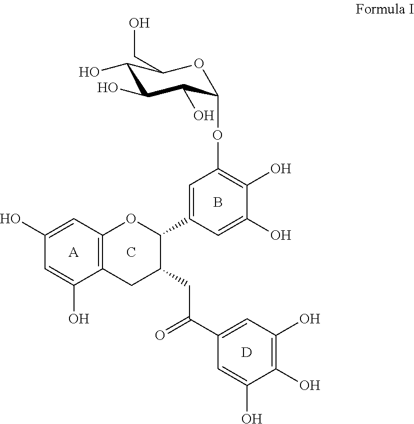

According to some embodiments, the carbohydrate derivative is a conjugate of a carbohydrate with a lipid. According to some embodiments, the carbohydrate derivative comprises a carbohydrate linked with a lipid. According to some embodiments, the saccharide is selected from a monosaccharide, disaccharide, trisaccharide, tetrasaccharide and oligosaccharide. According to some embodiments, the saccharide is selected from glucose, ribose, arabinose, galactose, mannose, sucrose and maltotriose. According to some embodiments, the lipid is selected from phospholipids, fatty acids, triglycerides and amino alcohol such as serine and hydroxyproline.

According to some embodiments, the phospholipid is selected from phosphatidylcholine, polyenylphosphatidylcholine, phosphatidylinositol, phosphatidylglycerol, phosphatidylethanolamine, 1-palmitoyl-2-oleoylphosphatidyl choline (POPC), sphingophospholipids, distearoyl, and any combination thereof. According to another embodiment, the liposome-forming lipid is a phospholipid. According to some embodiments, the amino alcohol is sphingosine. According to some embodiments, the glycosphingolipid is a ganglioside. According to some embodiments, the carbohydrate derivative is a glycosphingolipid. According to some embodiments, the glycosphingolipid is cerebroside. According to some embodiments, the glycosphingolipid is glucocerebroside. According to some embodiments, the cerebroside such as glucocerebroside comprises a nervonic acid as a lipophilic chain.

According to some embodiments, the carbohydrate derivative does not comprise cholesterol. According to some embodiments, the cargo molecule does not comprise cholesterol.

According to some embodiments, the active agent is directly bound to said carbohydrate or a derivative thereof.

According to any one of the aspects and embodiments, a carbohydrate is used as a loading agent, enhancer or provider of the active agent.

According to some embodiments, the active agent is bound to said carbohydrate or a derivative thereof via a linker. According to some embodiments, the linker is selected from hydrophilic, hydrophobic, and amphiphilic linkers. According to some embodiments, the linker is a DBCO-C6-Acid having CAS number 1425485-72-8.

According to some embodiments, the active agent is covalently bound to a carbohydrate or a derivative thereof via a cleavable bond or linker. According to some embodiments, the cleavage may be made via enzymatic reaction. According to some embodiments, the cleavage may be made via a chemical reaction.

According to some embodiments, the active agent is a nucleic acid. According to some embodiments, the active agent is an oligonucleotide. According to some embodiments, the active agent is a polynucleotide.

The term “nucleic acid” refers to a single-stranded or double-stranded sequence (polymer) of deoxyribonucleotides or ribonucleotides. In addition, the polynucleotide includes variants of natural polynucleotides, unless specifically mentioned. According to an embodiment, the nucleic acid may be” selected from the group consisting of deoxyribonucleic acid (DNA), ribonucleic acid (RNA), peptide nucleic acid (PNA), locked nucleic acid (LNA), and analogs thereof, but is not limited thereto. The term encompasses DNA, RNA, single-stranded or double-stranded and chemical modifications thereof.

The term “polynucleotide” as used herein refers to a long nucleic acid comprising more than 150 nucleotides.

The terms “nucleic acid” and “polynucleotide” are used interchangeably herein.

The term “oligonucleotide” as used herein refers to a short single-stranded or double-stranded sequence of nucleic acid such as ribonucleic acid (RNA), deoxyribonucleic acid (DNA) or mimetics thereof, said nucleic acid has typically less than or equal to 150 nucleotides.

According to some embodiments, the oligonucleotide comprises from 2 to 150, from 10 to 100, or 15 to 50 nucleotides.

According to some embodiments, the nucleic acid is selected from RNA, RNAi, siRNA, shRNA, saRNA, miRNA, and miRNA inhibitors. According to some embodiments, the nucleic acid is siRNA. According to some embodiments, the nucleic acid is shRNA.

According to some embodiments, the present invention provides isolated extracellular vesicles comprising a cargo molecule, wherein the cargo molecule comprises a nucleic acid molecule chemically bound to a carbohydrate or derivative thereof. According to some embodiments, the cargo molecule is an exogenous molecule. According to some embodiments, the nucleic acid molecule is siRNA. According to some embodiments, the nucleic acid molecule is shRNA. According to some embodiments, the carbohydrate is glucose. According to some embodiments, the carbohydrate is sucrose. According to some embodiments, the carbohydrate is fructose. According to some embodiments, the carbohydrate is arabinose. According to some embodiments, the nucleic acid is covalently bound to a carbohydrate. According to some embodiments, the nucleic acid is covalently bound to a carbohydrate via a linker. According to some embodiments, the linker is a DBCO-C6-acid. According to some embodiments, the nucleic acid is a nucleic acid and the carbohydrate is bound to its 5′ end. According to some embodiments, the nucleic acid is a nucleic acid and the carbohydrate is bound to its 3′ end. According to some embodiments, the nucleic acid is a siRNA and the carbohydrate is bound to its sense strand. According to some embodiments, the nucleic acid is a siRNA and the carbohydrate is bound to its anti-sense strand. According to some embodiments, the isolated extracellular vesicles comprise a cargo molecule as depicted in FIG. 4. According to some embodiments, the present invention provides isolated extracellular vesicles comprising siRNA chemically bound to a carbohydrate or derivative thereof. According to some embodiments, the siRNA or shRNA comprises the nucleic acid sequences AUCUAUAAUGAUCAGGUUCAU (SEQ ID NO: 1) and GAACCUGAUCAUUAUAGAU (SEQ ID NO: 2). According to some embodiments, the siRNA comprises the nucleic acid sequences SEQ ID NO: 1 and SEQ ID NO: 2 and the carbohydrate is bound to the 3′ of the sense strand, e.g. via a linker. According to some embodiments, the bond is a cleavable bond. According to some embodiments, the present invention provides isolated extracellular vesicles comprising a cargo molecule, wherein the cargo molecule comprises siRNA molecule covalently bound to glucose, optionally via a DBCO-C6-acid linker. According to some embodiments, the present invention provides isolated extracellular vesicles comprising a cargo molecule, wherein the cargo molecule comprises a siRNA molecule covalently bound to sucrose, optionally via a DBCO-C6-acid linker. According to some embodiments, the present invention provides isolated extracellular vesicles comprising a cargo molecule, wherein the cargo molecule comprises siRNA molecule covalently bound to arabinose, optionally via a DBCO-C6-acid linker.

According to some embodiments, from about 20 to about 100% of the EVs comprise the cargo molecules of the present invention. According to some embodiments, the cargo molecule is exogenous. According to some embodiments, from about 25% to about 95%, from about 30% to about 90%, from about 35% to about 85%, from about 40% to about 80%, from about 45% to about 75%, from about 50% to about 70%, or from about 55% to about 65% of the EVs comprise the cargo molecule of the present invention.

According to another aspect, the present invention provides a method of loading isolated extracellular vesicles (EVs) with cargo molecules, comprising incubating a population of EVs with cargo molecules comprising an active agent chemically bound to a carbohydrate or derivative thereof.

According to some embodiments, the active agent is selected from a small molecule, protein, peptide, polypeptide, lipid, carbohydrate and nucleic acid. According to some embodiments, the active agent is selected from a small molecule, protein, peptide, polypeptide, lipid, and nucleic acid. According to some embodiments, the active agent is selected from a small molecule, lipid, carbohydrate and nucleic acid.

According to some embodiments, the EVs are exosomes. According to some embodiments, the EVs are microvesicles. According to a further embodiment, the EVs are a combination of small and large vesicles.

According to any one of the above embodiments, the EVs are isolated. The EVs may be isolated from the cells by standard isolation and washing protocol by differential centrifugation, size exclusion or any other method for particles isolation protocol from the medium.

According to any one of the above embodiments and aspects, the carbohydrate is selected from a monosaccharide, disaccharide, trisaccharide, tetrasaccharide, and oligosaccharide.

According to some embodiments, the carbohydrate is a monosaccharide. According to some embodiments, the monosaccharide is selected from glucose, fructose ribose, arabinose, galactose, mannose and xylose. According to some embodiments, the monosaccharide is glucose. According to some embodiments, the monosaccharide is fructose. According to some embodiments, the monosaccharide is arabinose.

According to some embodiments, the carbohydrate is a disaccharide. According to some embodiments, the disaccharide is selected from sucrose, lactose and maltose. According to some embodiments, the disaccharide is sucrose.

According to some embodiments, the carbohydrate is a trisaccharide. According to some embodiments, the trisaccharide is selected from maltotriose and raffinose.

According to some embodiments, the carbohydrate is a tetrasaccharide.

According to some embodiments, the carbohydrate is an oligosaccharide.

According to some embodiments, the carbohydrate derivative is selected from a conjugate of a saccharide with an amino acid, a polyphenol, or lipid. The carbohydrate derivative is as described in any one of the above embodiments.

According to some embodiments, the active agent is directly bound to said carbohydrate or a derivative thereof.

According to some embodiments, the active agent is bound to said carbohydrate or a derivative thereof via a linker. According to some embodiments, the linker is selected from hydrophilic, hydrophobic, and amphiphilic linkers. According to some embodiments, the linker is 10-hydroxydecanoic acid.

According to some embodiments, the active agent is a nucleic acid. According to some embodiments, the nucleic acid is a oligonucleotide. According to some embodiments, the nucleic acid is selected from RNA, RNAi, siRNA, shRNA, saRNA, miRNA, and miRNA inhibitors. According to some embodiments, the nucleic acid is siRNA. According to some embodiments, the nucleic acid is shRNA.

According to some embodiments, the method of preparation of the EVs of the present invention comprises incubating a population of EVs with cargo molecules comprising nucleic acids selected from siRNA, shRNA, saRNA, miRNA, RNAi, or mRNA bound to a carbohydrate or derivative thereof selected from glucose, ribose, arabinose, galactose, mannose, sucrose, maltotriose, (-)-epigallocatechin gallate 3′-O-α-D-glucoside, isoquercitrin, isoquercetin, baicalin, puerarin, cerebroside and glucocerebroside. According to some embodiments, the method of preparation of the EVs of the present invention comprises incubating a population of EVs with cargo molecules, wherein the cargo molecules comprise a nucleic acid molecule chemically bound to a carbohydrate or derivative thereof. According to some embodiments, the cargo molecule is an exogenous molecule. According to some embodiments, the nucleic acid molecule is siRNA. According to some embodiments, the nucleic acid molecule is shRNA. According to some embodiments, the carbohydrate is glucose. According to some embodiments, the carbohydrate is sucrose. According to some embodiments, the carbohydrate is fructose.

According to some embodiments, the carbohydrate is arabinose. According to some embodiments, the nucleic acid is covalently bound to a carbohydrate. According to some embodiments, the nucleic acid is covalently bound to a carbohydrate via a linker. According to some embodiments, the linker is a DBCO-C6-acid. According to some embodiments, the nucleic acid is a nucleic acid and the carbohydrate is bound to its 5′ end. According to some embodiments, the nucleic acid is a nucleic acid and the carbohydrate is bound to its 3′ end. According to some embodiments, the nucleic acid is a siRNA and the carbohydrate is bound to its sense strand. According to some embodiments, the nucleic acid is a siRNA and the carbohydrate is bound to its anti-sense strand. According to some embodiments, the isolated extracellular vesicles comprise a cargo molecule as depicted in FIG. 4.

According to some embodiments, the present invention provides a method of preparation of isolated extracellular vesicles loaded with siRNA comprising incubating siRNA chemically bound to a carbohydrate or derivative thereof with isolated EVs. According to some embodiments, the siRNA or shRNA comprises the nucleic acid sequences AUCUAUAAUGAUCAGGUUCAU (SEQ ID NO: 1) and

GAACCUGAUCAUUAUAGAU (SEQ ID NO: 2). According to some embodiments, the siRNA comprises the nucleic acid sequences SEQ ID NO: 1 and SEQ ID NO: 2 and the carbohydrate is bound to the 3′ of the sense strand, e.g. via a linker. According to some embodiments, the linker is a DBCO-C6-acid. According to some embodiments, the bond is a cleavable bond. According to some of the above embodiments, the EVs are derived from adherent cells expressing mesenchymal markers. According to some of the above embodiments, the adherent cells expressing mesenchymal markers are mesenchymal stem cells (MSC). According to some of the above embodiments, the mesenchymal stem cells are human bone marrow mesenchymal stem cells. According to some of the above embodiments, the EVs are exosomes.

According to some embodiments, from about 20 to about 100% of the resulting EVs comprise the cargo molecules of the present invention. According to some embodiments, the cargo molecule is exogenous. According to some embodiments, from about 25% to about 95%, from about 30% to about 90%, from about 35% to about 85%, from about 40% to about 80%, from about 45% to about 75%, from about 50% to about 70%, or from about 55% to about 65% of the EVs comprises the cargo molecules of the present invention. According to some embodiments, from about 20 to about 100% from about 25% to about 95%, from about 30% to about 90%, from about 35% to about 85%, from about 40% to about 80%, from about 45% to about 75%, from about 50% to about 70%, or from about 55% to about 65% of the EVs are loaded with the cargo molecules of the present invention.

According to some embodiments, the method of the present invention further comprises electroporation or use of a transfection reagent such as a lipid transfection reagent.

According to alternative embodiments, the method of the present invention takes place in the absence of electroporation and of a transfection reagent.

According to some embodiments, the loading of EVs with the cargo molecules is performed/executed in the presence of insulin or derivatives thereof. According to some embodiments, the insulin is selected from Insulin aspart, Insulin glulisine, Insulin lispro, Insulin regular, NPH-insulin, Insulin detemir, Insulin glargine, Insulin degludec and mixtures thereof. According to some embodiments, the insulin is a fast-acting, intermediate-acting or long-acting insulin. According to some embodiment, insulin is present in the concentration of 1 to 1000 nM. According to some embodiment, insulin is present in the concentration of from 1 to 1000 U/ml. According to some embodiment, insulin is present in the concentration of from 10 to 1000 U/ml. According to some embodiment, insulin is present in the concentration of from 20 to 800 U/ml, from 30 to 700 U/ml, from 40 to 600 U/ml, from 50 to 500 U/ml, from 60 to 400 U/ml, from 70 to 300 U/ml, from 80 to 200 U/ml, from 90 to 150 U/ml, from 80 to 150 U/ml, from 70 to 120 U/ml, or from 80 to 120 U/ml. According to some embodiment, insulin is present in the concentration of from 0.1 to 1000 U/ml. According to some embodiment, insulin is present in the concentration of from 0.1 to 100 U/ml or from 0.5 to 50 U/ml. The insulin may present along the entire loading time (incubation of the EVs with the cargo molecules) or any part of that time.

According to some embodiments, insulin significantly increases the uptake of the cargo molecules of the present invention by EVs in comparison to EVs loaded without insulin. This is especially significant for cargo molecules comprising an active molecule bound to glucose. According to some embodiments, insulin increases the uptake of the cargo molecules into EVs by at least 10% in comparison to corresponding conditions that do not include (lacks or devoid of) insulin. According to some embodiments, insulin increases the uptake of the cargo molecules into EVs by at least 15%, at least 20%, at least 25%, at least 30%, at least 40%, at least 45%, at least 50%, at least 55%, at least 60%, at least 65%, at least 70%, at least 75% or at least 80% or at least 100% in comparison to corresponding conditions that do not include insulin. According to some embodiments, insulin increases the uptake of the cargo molecules of the present invention into EVs from by 10% to 60% in comparison to corresponding conditions that do not include insulin in the buffer during the loading. According to some embodiments, insulin increases the uptake of the cargo molecules into EVs by from 15 to 55%, from 20 to 50%, from 25 to 45%, from 30 to 50%, from 30 to 55%, from 35 to 50% or from 35 to 45% in comparison to corresponding conditions that do not include insulin. According to some embodiments, the cargo molecule comprises an active agent covalently bound to glucose. According to some embodiments, the amount of the cargo molecules in the resulting EVs is from 10 to 60%, from 15 to 55%, from 20 to 50%, from 25 to 45%, from 30 to 50%, from 30 to 55%, from 35 to 50% or from 35 to 45% more than in EVs loaded without insulin. According to some embodiments, the amount of the cargo molecules in the resulting EVs is at least 10%, at least 15%, at least 20%, at least 25%, at least 30%, at least 40%, at least 45%, at least 50%, at least 55%, at least 60%, at least 65%, at least 70%, at least 75% or at least 80% or at least 100% higher than in EVs loaded without insulin. The terms “substantially devoid”, “essentially devoid”, “devoid”, “does not include” and “does not comprise” may be used interchangeably and refer to a composition that does not include, contain or comprise a particular component, e.g. said composition comprises less than 0.1 wt %, less than 0.01 wt %, or less than 0.001 wt % of the component. According to some of the above embodiments, the EVs are derived from adherent cells expressing mesenchymal markers. According to some of the above embodiments, the adherent cells expressing mesenchymal markers are mesenchymal stem cells (MSC). According to some of the above embodiments, the mesenchymal stem cells are human bone marrow mesenchymal stem cells. According to some of the above embodiments, the EVs are exosomes.

According to another aspect, the present invention provides EVs obtainable or obtained by the methods of the present invention as described in any one of the above embodiments. According to some embodiments, the EVs comprise cargo molecules loaded by the methods of the present invention. According to some of the above embodiments, the EVs are derived from adherent cells expressing mesenchymal markers. According to some of the above embodiments, the adherent cells expressing mesenchymal markers are mesenchymal stem cells (MSC). According to some of the above embodiments, the mesenchymal stem cells are human bone marrow mesenchymal stem cells. According to some of the above embodiments, the EVs are exosomes.

According to yet another aspect, the present invention provides a composition comprising the EVs of the present invention, e.g. loaded with cargo molecules, and a carrier. According to some embodiments, the EVs are obtained or obtainable by the methods of the present invention.

The term “carrier” as used herein refers to as a class any compound or composition useful in facilitating storage, stability, administration, cell targeting and/or delivery of the topical composition, including, without limitation, suitable vehicles, skin conditioning agents, skin protectants, diluents, emollients, solvents, excipients, pH modifiers, salts, colorants, rheology modifiers, thickeners, lubricants, humectants, antifoaming agents, erodeable polymers, hydrogels, surfactants, emulsifiers, emulsion stabilizers, adjuvants, surfactants, preservatives, chelating agents, fatty acids, mono-di-and tri-glycerides and derivates thereof, waxes, oils and water.

According to some embodiments, the composition is a pharmaceutical composition and the carrier is a pharmaceutically acceptable carrier.

Thus, according to some embodiments, the present invention provides a pharmaceutical composition comprising a population of EVs according to any one of the above embodiments and aspects, and a pharmaceutically acceptable carrier. According to some embodiments, the present invention provides a pharmaceutical composition comprising a population of EVs obtained or obtainable by the methods of the present invention, and a pharmaceutically acceptable carrier.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well-known in the art. The compositions may contain other active compounds providing supplemental, additional, or enhanced therapeutic functions. Solid carriers or excipients may be for example, lactose, starch or talcum or liquid carriers such as, for example, water, fatty oils or liquid paraffins.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms and derivatives thereof such as, but not limited to, glycine, alanine, aspartic acid, glutamic acid, hydroxyproline, isoleucine, leucine and phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerin, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or another therapeutic agent(s), together with a suitable amount of carrier so as to provide the form for proper administration to the subject.

According to some embodiments, the pharmaceutical composition of the present invention comprises a population of EVs comprising cargo molecules, wherein the cargo molecules comprise a nucleic acid molecule chemically bound to a carbohydrate or derivative thereof. According to some embodiments, the cargo molecule is an exogenous molecule. According to some embodiments, the nucleic acid molecule is siRNA. According to some embodiments, the nucleic acid molecule is shRNA. According to some embodiments, the carbohydrate is glucose. According to some embodiments, the carbohydrate is sucrose. According to some embodiments, the carbohydrate is fructose. According to some embodiments, the carbohydrate is arabinose. According to some embodiments, the nucleic acid is covalently bound to a carbohydrate. According to some embodiments, the nucleic acid is covalently bound to a carbohydrate via a linker. According to some embodiments, the linker is a DBCO-C6-acid. According to some embodiments, the nucleic acid is a nucleic acid and the carbohydrate is bound to its 5′ end. According to some embodiments, the nucleic acid is a nucleic acid and the carbohydrate is bound to its 3 end. According to some embodiments, the nucleic acid is a siRNA and the carbohydrate is bound to its sense strand. According to some embodiments, the nucleic acid is a siRNA and the carbohydrate is bound to its anti-sense strand. According to some embodiments, the isolated extracellular vesicles comprise a cargo molecule as depicted in FIG. 4. According to some embodiments, the present invention provides isolated extracellular vesicles comprising siRNA chemically bound to a carbohydrate or derivative thereof. According to some embodiments, the siRNA or shRNA comprises the nucleic acid sequences AUCUAUAAUGAUCAGGUUCAU (SEQ ID NO: 1) and GAACCUGAUCAUUAUAGAU (SEQ ID NO: 2). According to some embodiments, the siRNA comprises the nucleic acid sequences SEQ ID NO: 1 and SEQ ID NO: 2 and the carbohydrate is bound to the 3′ of the sense strand, e.g. via a linker. According to some embodiments, the bond is a cleavable bond. According to some of the above embodiments, the EVs are derived from adherent cells expressing mesenchymal markers. According to some of the above embodiments, the adherent cells expressing mesenchymal markers are mesenchymal stem cells (MSCs). According to some of the above embodiments, the mesenchymal stem cells are human bone marrow mesenchymal stem cells. According to some of the above embodiments, the EVs are exosomes. According to some embodiments, the present invention provides a pharmaceutical composition comprising isolated extracellular vesicles comprising a cargo molecule, wherein the cargo molecule comprises a siRNA molecule covalently bound to glucose, optionally via a DBCO-C6-acid linker. According to some embodiments, the present invention provides a pharmaceutical composition comprising isolated extracellular vesicles comprising a cargo molecule, wherein the cargo molecule comprises a siRNA molecule covalently bound to sucrose, optionally via a DBCO-C6-acid linker. According to some embodiments, the present invention provides isolated extracellular vesicles comprising a cargo molecule, wherein the cargo molecule comprises a siRNA molecule covalently bound to arabinose, optionally via a DBCO-C6-acid linker.

According to some embodiments, the pharmaceutical composition is for use in treating and/or preventing a disease, disorder or condition treatable with the active agent loaded into the EVs. It is clear that the use depends on the molecule loaded in the EVs and will be adapted accordingly. As such, a pharmaceutical composition comprising EVs loaded with siRNA inhibiting expression of Phosphatase and tensin homolog PTEN) protein, is for use in treating any disease or condition in which reduction of PTEN protein expression is required, such as neurodegenerative disease, neuronal disorder, neuronal injury, CNS damage, neuronal injury or damage is a spinal cord injury (SCI).

The term “treating” a condition or patient refers to taking steps to obtain beneficial or desired results, including clinical results. Beneficial or desired clinical results include, but are not limited to, ameliorating, abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

As used herein, the term “preventing” when used in relation to a condition, refers to the administration of a composition that reduces the frequency of, or delays the onset of, symptoms of a medical condition in a subject relative to a subject which does not receive the composition.

The pharmaceutical composition of the present invention may be administered using any known method. The terms “administering” or “administration of” a substance, a compound or an agent to a subject can be carried out using one of a variety of methods known to those skilled in the art. For example, a compound or an agent can be administered intranasally (e.g., by inhalation), intrathecally (into the spinal canal, or into the subarachnoid space), arterially, intradermally, intramuscularly, intraperitoneally, intravenously, subcutaneously, ocularly, sublingually, orally (by ingestion), intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition of the present invention is administered intranasally. According to another embodiment, the pharmaceutical composition of the present invention is administered intra-lesion. According to another embodiment, the pharmaceutical composition of the present invention is administered in proximity to the damage or injury. According to one embodiment, the pharmaceutical composition is administered orally.

According to one embodiment, the pharmaceutical composition is administered intranasally. According to some embodiments, the pharmaceutical composition is administered locally. According to some embodiments, the pharmaceutical composition is administered systemically.

According to another aspect, the present invention provides a method of delivering an active agent comprising exposing a mammal, organ, tissue, or target cell to EVs of the present invention comprising cargo molecules of the present invention comprising the active agent, e.g. those obtained or obtainable by the methods of the present invention.

According to yet another aspect, the present invention provides a method of treating a disease, medical condition or disorder treatable by an active agent, the method comprises administering to a subject in need thereof a therapeutically effective amount of EVs of the present invention comprising cargo molecules comprising the active agent bound to a carbohydrate, as described in any one of the above embodiments.

According to another aspect, the present invention provides a conjugate molecule comprising an exogenous nucleic acid chemically bound to a carbohydrate or derivative thereof. All terms and embodiments defined above apply and are encompassed herein as well. According to some embodiments, the conjugate molecule is devoid of cholesterol.

According to some embodiments, wherein the carbohydrate is selected from a monosaccharide, disaccharide, trisaccharide, tetrasaccharide and oligosaccharide and wherein the carbohydrate derivative is selected from a saccharide linked to an amino acid, polyphenol, or lipid.

According to one embodiment, the monosaccharide is selected from glucose, ribose, arabinose, galactose, mannose, fructose and xylose; the disaccharide is selected from sucrose, lactose and maltose. According to one embodiment, the trisaccharide is selected from maltotriose and raffinose. According to some embodiments, the saccharide linked to an amino acid is ribose-cysteine. According to some embodiments, the saccharide linked with a polyphenol is selected from (-)-epigallocatechin gallate 3′-O-α-D-glucoside, isoquercitrin, baicalin and puerarin. According to some embodiments, the saccharide linked with a lipid is a cerebroside. According to some embodiments, the cerebroside is glucocerebroside.

According to some embodiments, the nucleic acid is bound to a carbohydrate or derivative thereof directly or via a linker. Any linker, e.g. those defined hereinabove, may be used. According to some embodiments, the nucleic acid is an oligonucleotide.

According to some embodiments, the oligonucleotide is selected from RNA, RNAi, siRNA, shRNA, miRNA, miRNA inhibitor, and short activating RNA (saRNA). Thus, according to some embodiments, the present invention provides a conjugate comprising an oligonucleotide selected from RNA, RNAi, siRNA, shRNA, miRNA, and miRNA inhibitor chemically bound to a carbohydrate or derivative thereof. According to some embodiments, the nucleic acid molecule is siRNA. According to some embodiments, the nucleic acid molecule is shRNA. According to some embodiments, the carbohydrate is glucose. According to some embodiments, the carbohydrate is sucrose. According to some embodiments, the carbohydrate is fructose. According to some embodiments, the carbohydrate is arabinose. According to some embodiments, the nucleic acid is covalently bound to a carbohydrate. According to some embodiments, the nucleic acid is covalently bound to a carbohydrate via a linker. According to some embodiments, the linker is a DBCO-C6-acid linker. According to some embodiments, the nucleic acid is a nucleic acid and the carbohydrate is bound to its 5′ end. According to some embodiments, the nucleic acid is a nucleic acid and the carbohydrate is bound to its 3′ end. According to some embodiments, the nucleic acid is a siRNA and the carbohydrate is bound to its sense strand. According to some embodiments, the nucleic acid is a siRNA and the carbohydrate is bound to its anti-sense strand. According to some embodiments, the cargo molecule has a structure as depicted in FIG. 4. According to some embodiments, the present invention provides isolated extracellular vesicles comprising siRNA chemically bound to a carbohydrate or derivative thereof. According to some embodiments, the siRNA or shRNA comprises the nucleic acid sequences AUCUAUAAUGAUCAGGUUCAU (SEQ ID NO: 1) and GAACCUGAUCAUUAUAGAU (SEQ ID NO: 2). According to some embodiments, the siRNA comprises the nucleic acid sequences SEQ ID NO: 1 and SEQ ID NO: 2 and the carbohydrate is bound to the 3′ of the sense strand, e.g. via a linker. According to some embodiments, the bond is a cleavable bond. According to some embodiments, the active agent is covalently bound to a carbohydrate or a derivative thereof via a cleavable bond or linker. According to some embodiments, the cleavage may be made via enzymatic reaction. According to some embodiments, the cleavage may be made via a chemical reaction. According to some embodiments, the present invention provides an exogenous siRNA molecule covalently bound to glucose, optionally via a DBCO-C6-acid linker. According to some embodiments, the present invention provides an exogenous siRNA molecule covalently bound to sucrose, optionally via a DBCO-C6-acid linker. According to some embodiments, the present invention provides an exogenous siRNA molecule covalently bound to arabinose, optionally via a DBCO-C6-acid linker.

The terms “a,” “an,” and “the” are used herein interchangeably and mean one or more.

The term “and/or” is used to indicate one or both stated cases may occur, for example, A and/or B includes, (A and B) and (A or B).

The term “or,” as used herein, denotes alternatives that may, where appropriate, be combined; that is, the term “or” includes each listed alternative separately as well as their combination if the combination is not mutually exclusive.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

Having now generally described the invention, the same will be more readily understood through reference to the following examples, which are provided by way of illustration and are not intended to be limiting of the present invention.

### EXAMPLES

### Example 1

The loading protocol is co-incubation of 3 different concentrations of each of the saccharides, saccharide-derivatives, saccharide-derivatives-siRNA conjugates and saccharides-siRNA conjugates with 10⁶-10⁸ exosomes per μl at 25° C. and 37° C. for 2 and 4 hours with and without insulin. The tested saccharides and saccharide-derivatives are glucose, ribose, arabinose, galactose, sucrose, mannose, maltotriose, (-)-epigallocatechin gallate 3′-O-α-D-glucoside, isoquercitrin, isoquercetin, baicalin, puerarin, cerebroside and glucocerebroside. All possibilities are tested in triplicates. Post incubation, the exosomes are separated from the medium and washed twice. The loaded exosomes are used for quantifying intra-exosomes saccharide, saccharide-derivatives, saccharides-siRNA conjugates, and siRNA concentrations, and later also for relevant in vitro experiments.

The intra-exosomes saccharide/saccharides-derivatives, saccharides/saccharides-derivatives-siRNA conjugates, and siRNA concentrations are tested using at least two different analytical methods (e.g., using fluorescent labeling, ELISA, WB, PCR, LC-MS/MS).

Exosomes loaded by the most successful cargo molecules comprising saccharides/saccharide derivatives, and saccharides/saccharide derivatives bound to siRNA (that achieved the highest intra-exosome concentrations) are then incubated with cells to demonstrate suppression and inhibition of the relevant gene and protein.

To determine the efficiency of the saccharide/saccharides-derivatives, saccharides/saccharides-derivatives-siRNA conjugates for the loading of the exosomes the following procedures is be performed.

After the loading protocol of co-incubation of 3 different concentrations of each of the saccharides, saccharide-derivatives, saccharide-derivatives-siRNA conjugates, and saccharides-siRNA conjugates with 10⁶-10⁸ exosomes per μl at 25° C. and 37° C. for 2 and 4 hours, cy-3 labeled siRNA will be added to each sample to determine the efficiency of the loading. Exosomes will be labeled with a fluorescent dye for the exosome membrane staining (as Carboxyfluorescein succinimidyl ester-CFSE green dye), so the cell membrane will be stained with the CFSE green dye and the siRNA cargo with the Cy3/FAM red labeling. Using the Cytoflex/Nanosight or other nanoparticle tracking analysis technology (NTA) we will be able to see the loaded exosomes and to determine the loading efficiency for each condition.

In addition, the labeled loaded exosomes for each condition are added to the cells. Cells are observed under a confocal microscope to determine the proper engulfment of the exosomes into the cells.

Then, to evaluate the efficient suppression and inhibition of the siRNA-targeted genes with the siRNA loaded into exosomes, cells are incubated for 24 to 72 hours with the loaded exosomes from all the above-mentioned conditions, harvested, and tested at the RNA and protein levels. For the evaluation of the gene expression in the cells, RNA is extracted using the RNeasy mini kit (QIAGEN) according to the manufacturer's protocol. cDNA is prepared using the High-Capacity cDNA Reverse Transcription Kit (Thermo Fisher). Real-time quantitative PCR is conducted on the QuantStudio 12K Flex real-time PCR system using primers specific to the targeted gene. The AACt method is used to determine relative expression levels, where the gene of interest will be normalized to GAPDH expression.

For the evaluation of protein expression, cells are incubated for 24 to 72 hours with the loaded exosomes from all the above-mentioned conditions, harvested, and lysed. Lysates are tested for protein expression using either a western blot analysis or/and ELISA with antibodies specific to the targeted protein.

### Example 2

Four different saccharides (three monosaccharides and one disaccharide (glucose, mannose, galactose and sucrose) were incubated with extracellular vesicles derived from BM MSCs and following incubation, separated on UHPLC using special sugars Column: Phenomenex (Luna Omega) Agilent 1290 Infinity II combined with 1260 Infinity ELSD.

### Material and Methods

Glucose working solution—concentration of 1000PPM (˜1 mg/ml)
Sucrose working solution—concentration of 1000PPM (˜1 mg/ml)
Arabinose working solution—concentration of 1000PPM (˜1 mg/ml).
Galactose working solution—concentration of 1000PPM (˜1 mg/ml)
10 μl of EVs at a concentration of ˜10⁷ particles/μl (as determined on Nanosight. NS300) were added to each one of the four different saccharides solutions
Volume was completed to 100 μl using DDW.
The solutions were incubated while shaking for 30 min in RT.

### HPLC Parameters for Different Saccharides Separation

The separation was done in UHPLC using special sugars Column: Phenomenex (Luna Omega) Agilent 1290 Infinity II combined with 1260 Infinity ELSD

### Phase Making:

Water (A)
Mixture of solvents (B): Acetonitrile (90%) Isopropanol (5%), water (5%)
   The initial concentration of phase was 5% (A) and 95% (B)
   The concentration was gradually changed during the running, to 10% (A) and 90% (B)

The chromatographs of each saccharide, of EVs and the saccharide with EVs were analyzed and compared and the level of absorption of the saccharide into EVs was calculated. The fraction of the unbound saccharide is summarized in Table 1.

TABLE 1
The percentage of unbound saccharide

| Standard deviation | % leftover saccharides | Saccharide |
|---|---|---|
| 0 | 19 | Sucrose |
| 2 | 28 | Galactose |
| 1 | 20 | Glucose |
| 1 | 20 | Arabinose |

In this experiment, we examined the interaction of 4 different saccharides in fixed concentration (1000 ppm) and fixed concentration of exosome 10⁷ particles/82 l. The results show that in these conditions, up to 80% of each one of the saccharides were absorbed into/on the MSC-derived EVs.

In a similar arrangement, additional saccharides are tested: ribose, mannose, lactose, maltose maltotriose and raffinose.

### Example 3. Quantitative Measurement of Glucose Absorption Efficacy to BM MSC-Derived EVs

### Experiment Goals

The goal of this experiment was to quantitatively measure the glucose absorption efficacy to BM MSCs derived EVs and to develop an analytical method for QC and QA for quantifying glucose-conjugated molecules as loading reagents for EVs.

### Experiment Planning:

This experiment was based on previous results obtained in Example 2, showing a high absorption efficacy of four different saccharides (glucose, sucrose, mannose, and galactose) to BM MSCs derived EVs. Here, the focus was on glucose as a loading reagent to EVs, and on determining its absorption curve.

### Working Protocol

Glucose working solution—concentration of 1000 PPM
10 μl of EVs at a concentration of ˜10⁷ particles/μl (as determined on Nanosight NS300) were added to different glucose concentrations solutions (see Table 2)
Volume was completed to 100 μl using DDW.
The solution was incubated while shaking for 30 min in RT.

TABLE 2
Preparation of the solutions

| Glucose | Glucose | EVs (1*10E7 | |
|---|---|---|---|
| conc. in | solution | part/μl) | DDW |
| the mix (ppm) | Volume (μl) | Volume (μl) | Volume (μl) |
| 0 | 0 | 10 | 90.0 |
| 250 | 2.5 | 10 | 87.5 |
| 500 | 5.0 | 10 | 85.0 |
| 750 | 7.5 | 10 | 82.5 |
| 1000 | 10.0 | 10 | 80.0 |

All conditions were done in triplicates

### HPLC Parameters for Glucose Separation

The separation was done in UHPLC using special sugars Column: Phenomenex (Luna Omega) Agilent 1290 Infinity II combined with 1260 Infinity ELSD

### Phase Making:

Water (A)
Mixture of solvents (B): Acetonitrile (90%) Isopropanol (5%), water (5%)
The initial concentration of phase was 5% (A) and 95% (B)
The concentration was gradually changed during the running, to 10% (A) and 90% (B)

The integral of the chromatographs with different glucose concentrations (PPM) conditions and different controls were calculated to determine the amount of the absorbed glucose.

### Calibration Curve

As a first step, we validated that glucose concentration of 250 PPM to 1000 PPM is in the linear range of detection. A calibration curve was built and shows that this is the case with R²=0.9932. Further, absorption of glucose to exosomes was measured. The results are presented in Table 3.

### Absorption Curve

TABLE 3

| Glucose concentration | Mean absorbed glucose (%) | STD |
|---|---|---|
| 250 | 42 | 1.7 |
| 500 | 37 | 0.3 |
| 750 | 28 | 4.6 |
| 1000 | 9 | 5.3 |

The absorption capacity of Glucose to the EVs (UHPLC, Agilent) was ˜40% for glucose concentration of 250 PPM.

### Example 4: Quantitative Measurement of Sucrose Absorption Efficacy to BM MSC-derived EVs

### Experiment Goals:

The goals of this experiment were to measure sucrose absorption efficacy to BM MSC EVs in quantitative measurement, develop an analytical method for quantifying the loading of sucrose-conjugated molecules to exosomes, and to demonstrate that conjugating molecules with glucose is an efficient method for loading EVs with the required molecules. Different sucrose concentrations were used to measure the efficacy of absorption of disaccharide (in comparison to monosaccharide).

### Experiment Planning:

This experiment is based on previous results showing high absorption efficacy of four different monosaccharides (Glucose, Sucrose, Arabinose, Galactose) to MSC EVs. In this experiment, we focused on sucrose as a loading reagent to EVs, and define its absorption curve.

### Working Protocol

Sucrose solution was prepared in the concentration of 1000 PPM
10 μl of exosomes in a concentration of ˜10⁷ particles/μl [protein 3.01 μg/μl] were added to different sucrose concentrations solutions (see table below)
The solution was incubated while shaking for 0.5 h in room temperature.
The working concentrations are summarized in Table 4.

TABLE 4
Summary of concentrations

| Sucrose conc. | Sucrose | Exosomes | |
|---|---|---|---|
| in the mix | solution | (10⁷ part/μl) | DDW |
| (ppm) | Volume (μl) | Volume (μl) | Volume (μl) |
| 0 | 0 | 10 | 90.0 |
| 250 | 2.5 | 10 | 87.5 |
| 500 | 5.0 | 10 | 85.0 |
| 1000 | 10.0 | 10 | 80.0 |
| 1500 | 12.5 | 10 | 77.5 |

All conditions were done in triplicates

### HPLC Parameters for Sucrose Separation

The separation was done in UHPLC using special sugars Column: Phenomenex (Luna Omega) Agilent 1290 Infinity II combined with 1260 Infinity ELSD

### Phase Making:

Water (A)
Mixture of solvents (B): Acetonitrile (90%) Isopropanol (5%), water (5%)
The initial concentration of phase is 5% (A) and 95% (B)
The concentration is gradually changed during the running, to 10% (A) and 90% (B)

### Results

First, as in the case of glucose, we built a calibration plot to verify that the concentration 250-1500 PPM of sucrose is in the linear range of detection. The results are summarized in Table 5.

TABLE 5
Sucrose linearity curve

| ppm | Area 1 | Area 2 | Area 3 | Average | SD |
|---|---|---|---|---|---|
| 250 | 21.0 | 19.4 | 19.0 | 19.8 | 0.9 |
| 500 | 47.0 | 44.0 | 30.0 | 40.3 | 7.4 |
| 1000 | 92.5 | 85.7 | 93.3 | 90.5 | 3.4 |
| 1500 | 143.9 | 152.0 | 133.5 | 143.1 | 7.6 |

Sucrose in different concentrations shows a linear curve. This control is important to demonstrate the calibration of the system.

### EVs Validation

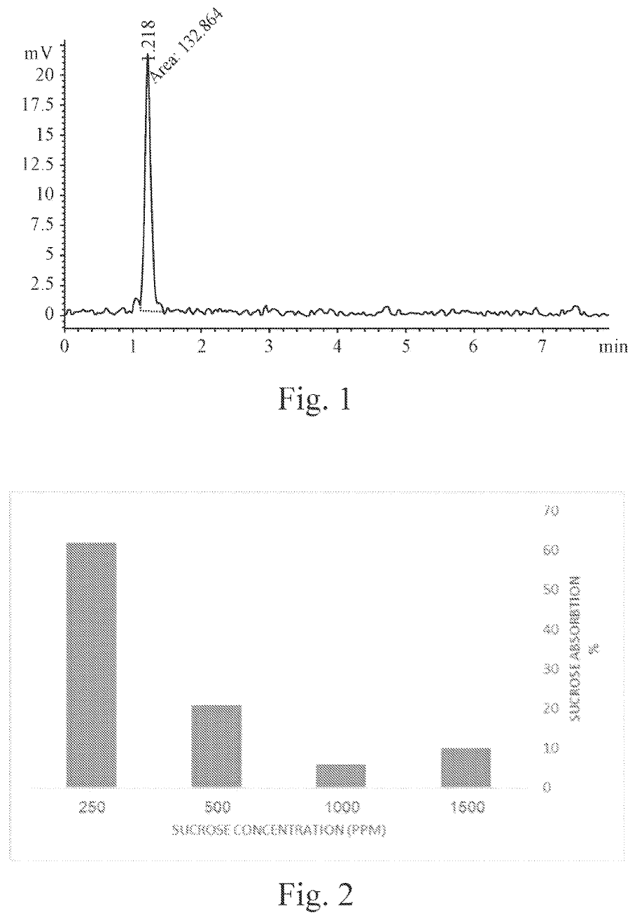

EVs alone were tested in UHPLC in order to validate them before the absorption test. The chromatogram shown below confirms their validity (FIG. 1).

### Analysis of Sucrose Absorption Curves

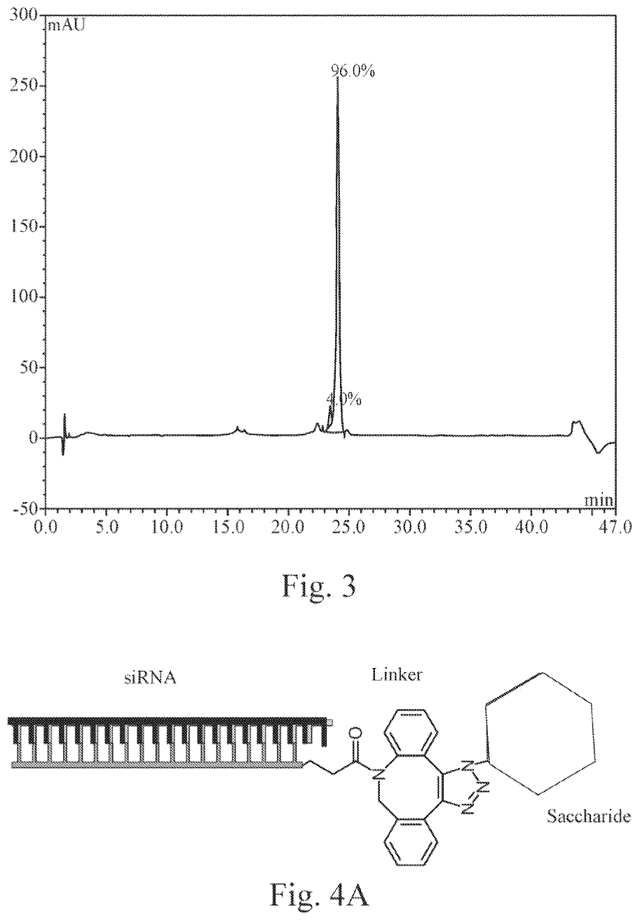

The results of the absorption of sucrose on EVs is presented in Table 5 and FIG. 2

TABLE 5

| Absorption of | | | | | |
|---|---|---|---|---|---|
| sucrose on EVs. | Area | Area | % absorbed | | |
| Sucrose Conc. | Exo | Sucrose | sucrose | Average | SD |
| 250 ppm | | | | | |
| 1 | 351 | 4 | 78 | | |
| 2 | 113 | 8 | 58 | | |
| 3 | 130 | 10 | 48 | 62 | 12.6 |
| 500 ppm | | | | | |
| 1 | 121 | 32 | 22 | | |
| 2 | 124 | 31 | 22 | | |
| 3 | 97 | 33 | 18 | 21 | 1.8 |
| 1000 ppm | | | | | |
| 1 | 132 | 75 | 17 | | |
| 2 | 107 | 83 | 8 | | |
| 3 | 136 | 97 | −7 | 6 | 9.9 |
| 1500 ppm | | | | | |
| 1 | 122 | 117 | 18 | | |
| 2 | 115 | 132 | 8 | | |
| 3 | 96 | 137 | 4 | 10 | 6.0 |

### Summary and Conclusions:

Altogether the results demonstrate an absorption curve with a ceiling absorbance of around 500 ppm sucrose.

### Example 5

The siRNA used for this experiment is anti-PTEN-siRNA1962 conjugated with a saccharide, more specifically with glucose. The siRNA sequence is described below.

| | Antisense | SEQ- | | |
|---|---|---|---|---|
| siRNA duplex | (A)/ | ID | Sequence | 3′ |
| ID | Sense (S) | No | (5′-3′) | modification |
| sirna_1962 | A | 1 | AUCUAUAAUGAUCAGGUUCAU | |
| | S | 2 | GAACCUGAUCAUUAUAGAU | -Linker- |
| | | | | glucose |

HPLC analysis of the siRNA is shown in FIG. 3. A schematic representation of such a conjugate is shown in FIG. 4A and a more specific conjugate with glucose is presented in FIG. 4B.

In other examples of the current invention, the siRNA is conjugated with other saccharides: sucrose, arabinose, mannose, galactose, ribose, lactose maltose maltotriose and raffinose.

### Example 6

As a next step, it was tested whether the conjugation of siRNA with glucose affects the efficacy of siRNA. It was done using regular transfection of the cells (by using Lipofectamine) exploiting conjugates as described in Example 5.

### Transfection:

Grow HEK293 to 90% confluency. One day before transfection, plate 20K cells/well in a 96-well plate with 100 μl of growth medium in each well. Transfection is performed in triplicates. Mix siRNAs with transfection media (Tmedia-growth media w/o serum) according to the manufacturer's protocol. Mix Lipofectamine™ RNAiMAX gently before use, then dilute according to the above. Mix gently. Combine the diluted RNAi duplex with the diluted Lipofectamine™ RNAiMAX. Mix gently and incubate for 5-20 minutes at room temperature.

Change media to all wells to be transfected with 100 μL growth media.

Add the RNAi duplex-Lipofectamine™ RNAiMAX complexes to each well-containing cell. Mix gently by rocking the plate back and forth. Incubate the cells for 24 hours at 37° C. 24 hours later, change media to all samples (100 μL media/well) and image cells under a fluorescent microscope to document transfection efficiency (pic/calculations). 72 hours later, cells are ready to be harvested for RNA purification.

### Gene Expression:

RNA was isolated with RNeasy Mini Kit as per the manufacturer's instructions. cDNA was prepared from 500 ng RNA with cDNA Reverse Transcription Kit (Applied Biosystems) as per the manufacturer's instructions

RNA levels were evaluated with designed Taqman probes. The delta Ct method was used to calculate a relative expression of the target gene (PTEN) in comparison to GAPDH as a normalizing gene. Expression levels were normalized to cells that were not transfected.

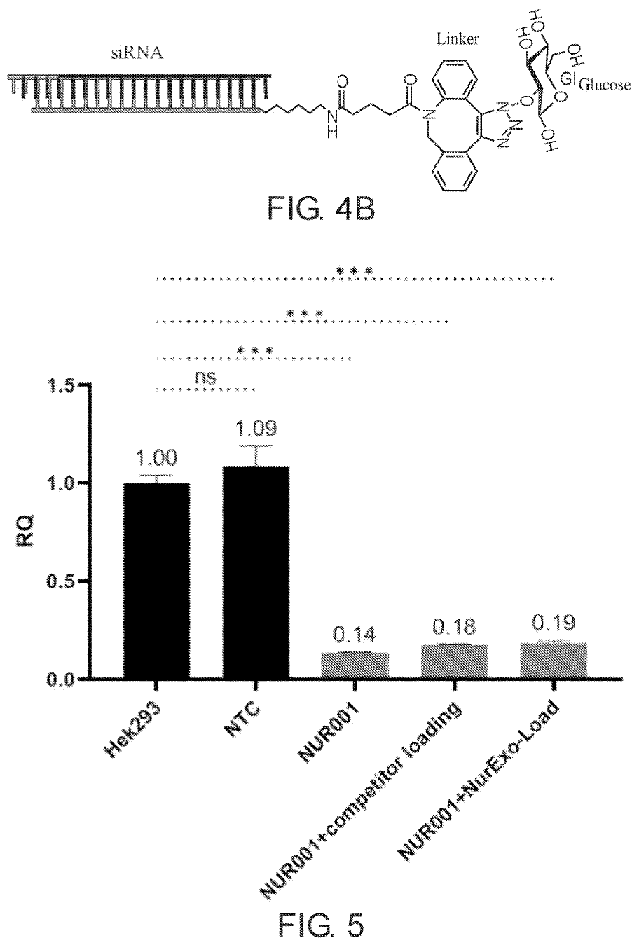

The results are presented in FIG. 5 and show that the conjugation of siRNA with glucose does not affect the siRNA's activity. In the figure: NUR001 is anti-PTEN siRNA; competitor loading—the conjugate of the siRNA with cholesterol; NurExo-Load—the conjugate of the siRNA with glucose as described in Example 4. As clearly seen in the figure, glucose conjugated to the siRNA does not affect the efficiency of the siRNA to knock down PTEN expression following transfection of HEK293 cells.

### Example 7

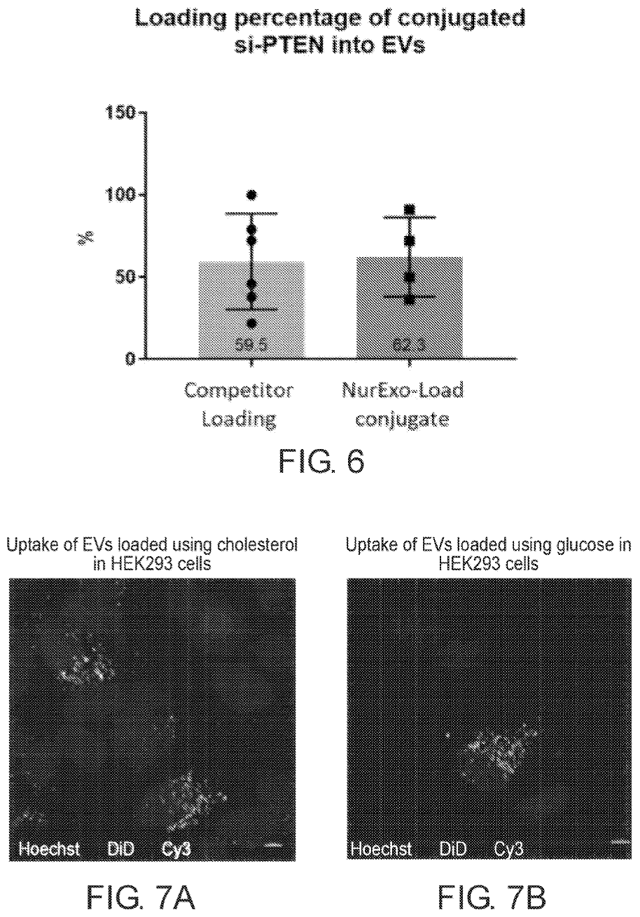

The objective of the experiment was to determine the loading efficiency of siRNA molecules into EVs, the percentage of EVs loaded with siRNA conjugated with a saccharide. SiRNA conjugated with glucose (as in Example 5) and cholesterol were compared. The EVs derived from bone marrow MSCs were stained with lipophilic dye (Protocol EV stain) and loaded with fluorescently labeled siRNA conjugates. A 35 nm qEV single column (Izon) was used for EVs' purification. The samples were eluted with filtered PBS (0.02 μm). The fractions collected were then transferred to amicon ultra centrifugal filter tubes with 30 kDa cutoff, the volume was then completed with PBS (0.02 μm) to 500 μl. The tubes were centrifuged at 14,000×g, RT for 10 min in order to concentrate the EVs. In order to obtain maximum recovery of the concentrate, the amicon ultra centrifugal filter tube was then inversed in a new collection tube and centrifuged at 1000×g, RT for 5 minutes. The fractions were then observed under high-resolution microscope and analyzed (data not shown). FIG. 6 shows the efficacy of loading of siRNA conjugated with glucose in comparison to the same siRNA conjugated with cholesterol. As can be seen from the figure, the efficacy of loading was about 60%, which is similar to loading with cholesterol conjugate.

In a similar arrangement, the siRNA conjugated with other saccharides: sucrose, arabinose, galactose ribose, mannose, lactose maltose maltotriose and raffinose is tested.

### Example 8

In this example, the ability of EVs loaded with glucose-conjugated or cholesterol-conjugated siRNA to enter cells was tested. For this purpose, the loaded EVs were incubated with human progenitor cells.

Method: ReN cells were grown to 80-90% confluency. One day before EV treatment, 10K cells/well were seeded in a Matrigel-coated 96well plates with 100 μl of growth medium. On the day of the experiment, media was replaced.
EVs were thawed and treated:
1. EVs stained with membranal marker (DiD) loaded with Ellap5-Cholesterol-Cy3
2. EVs stained with membranal marker (DiD) loaded with Ellap5-Glucose-Cy3
7 μL of each sample was added to each well in triplicates:
Cells were incubated for 24 hours at 37° C.

The next day, cells were washed once with PBS and Hoechst staining was performed. Images were obtained using a super-resolution microscope (Light Sheet, Zeiss Z7).

EVs loaded with glucose-conjugated or cholesterol-conjugated siRNA were similarly uploaded by cells, as can be seen in FIG. 7A and 7B.

In a similar arrangement, the siRNA conjugated with other saccharides: sucrose, arabinose, galactose ribose, mannose, lactose maltose maltotriose and raffinose is tested.

### Example 9. Loading of EVs in The Presence of Insulin

### Experiment I

Exosomes (200 μl of PBS; 10⁸ particles/μl) is incubated with 5 nmol siRNA conjugated with glucose, and 0.1 U of insulin added to the abovementioned 200 ul for 4 hours in 37 C. Next, the exosomes are washed by amicon filtration or by ultracentrifugation (100,000×G, 2h) and re-suspended with 200 μl of saline for further characterization. These results are compared to the control conditions, i.e. without addition of insulin. Following the addition of the loaded exosomes to cells, the comparison is made based on the expression of the silenced gene (i.e. PTEN) by RT-qPCR and western blot of the targeted gene, in the cells.

### Experiment II

Exosomes (20 μl of 10⁸ particles/μl) are incubated with 5 nmol siRNA bound to glucose or another (abovementioned reagent) and cy3/FAM as fluorescent labeling, and 10 μg of insulin for 2-4 hours in RT (or 37 C). Next, the exosomes are washed by amicon filtration or by ultracentrifugation (100,000G, 2 h) and re-suspended with 200 μl of saline for characterization. These results are compared to the control conditions of: exosomes incubated with siRNA conjugated with glucose without insulin. The output is a fluorescent analysis of the exosomes using Nanosight/Cytoflex or high-resolution microscopy.

### Experiment III

Extracellular Vesicles (EVs) obtained from bone marrow Mesenchymal stem cells (MSCs) (1×10¹⁰ particles/ml), were pre-incubated with Insulin (100 U/ml) at a dilution of 1:6000 for 20 minutes at 37° C. and then mixed with glucose as 2-NBDG (0.1 mM) (Invitrogen cat #N13195) (CTRL),at 37° C. for an hour. Afterward, the EVs were labeled with 1,1-Dioctadecyl-3,3,3,3-tetramethylindodicarbocyanine (DiD). To eliminate small particles, including dye aggregates, the EVs were eluted through an Izon column. EV suspension was visualized using a super-resolution microscope, and the fluorescent signal was measured using IMARIS software. The values are presented as mean±standard error of the mean (SEM).

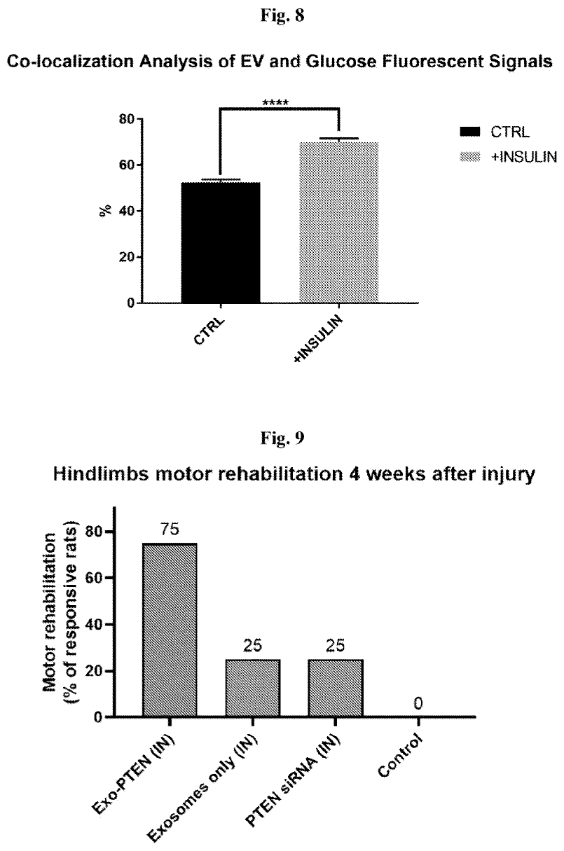

The results are presented in FIG. 8. It can be seen that the presence of insulin significantly increases the absorption of glucose into EVs, by about 40%.

Similar experiments are performed with saccharide such as sucrose, arabinose, galactose, ribose, mannose, lactose, maltose, maltotriose and raffinose.

Example 10. Treatment of full spinal cord injury with EVs loaded with siPTEN-glucose

In this experiment rats with full spinal cord injury were intranasally treated using EVs loaded with siPTEN-glucose.

The surgery and treatment administration were performed as described by Guo et al, 2019 ACS Nano 2019 13 (9), 10015-10028 DOI: 10.1021/acsnano.9b01892.

To assess the neuroprotective potential of ExoPTEN treatment, rats with complete SCI were divided into 4 groups (4 rats in each group at the beginning of the experiment): (1) no treatment, (2) PTEN-siRNA treatment, (3) exosomes only and (4) ExoPTEN. The treatment was given intranasally and initiated 2-3 h postinjury.

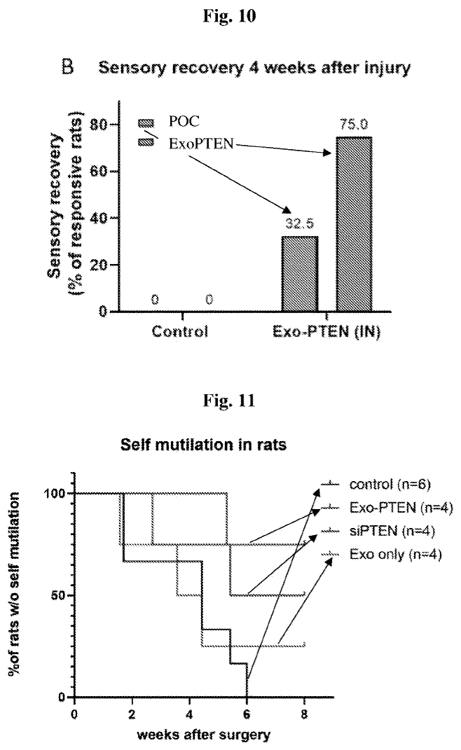

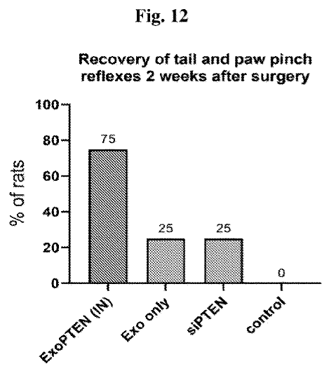

The results of the experiment show that Intranasal ExoPTEN treatment improves locomotor, sensory, and reflexes recovery and well-being in injured rats. Results of in vivo studies of spinal cord injury (full transection) in rats following intranasal proprietary ExoPTEN treatment are presented in FIGS. 9-12 In the figures, ExoPTEN is the EVs derived from bone marrow MSC loaded with siRNA_1962 conjugated with glucose, siPTEN refers to siRNA_1962; POC exosomes loaded with commercially available anti-PTEN siRNA conjugated with cholesterol having the sequences: antisense UUCUGUUUGUGGAAGAACUC (SEQ ID NO: 3) and sense GAGUUCUUCCACAAACAGAA (SEQ ID NO: 4); and control-saline.

The study showed that:
In the ExoPTEN-treated group comprising four rats that received intranasal administration of ExoPTEN, 75% of the rats responded to treatment and recovered hind limb reflex, rehabilitated some motor function, demonstrated no sign of self-harm (an indicator of stress) and recovered sensory control.
In the exosome-only and therapeutic PTEN siRNA molecule-only treated groups, each with four rats, 25% of the subjects experienced recovery of hind limb reflex, motor function, and sensory control, while also showing no signs of stress-related self-harm.
In a control group of six rats treated with a non-therapeutic saline solution, none exhibited any sensory, reflex recovery, or motor rehabilitation. All rats in this group displayed self-harm behavior, indicating a higher level of stress.

In addition, MRI analysis of the rats' spinal cords showed clearly the existence of tissue regeneration caudal to the T10 epicenter of the injury in treated rats compared to control (saline) rats.

Although the present invention has been described hereinabove by way of preferred embodiments thereof, it can be modified, without departing from the spirit and nature of the subject invention as defined in the appended claims.

1. Isolated extracellular vesicles comprising an exogenous cargo molecule, wherein the exogenous cargo molecule comprises an active agent chemically bound to at least one carbohydrate or derivative thereof, wherein the active agent is selected from a small molecule, protein, peptide, polypeptide, lipid, and a nucleic acid.

2. (canceled)

3. The isolated EVs according to any claim 1, wherein the carbohydrate is selected from a monosaccharide, disaccharide, trisaccharide, tetrasaccharide and oligosaccharide or wherein the carbohydrate derivative is selected from a saccharide linked to an amino acid, polyphenol, or lipid.

4. The isolated EVs according to claim 3, wherein the monosaccharide is selected from glucose, ribose, arabinose, galactose, mannose, and xylose; the disaccharide is selected from sucrose, lactose and maltose; the trisaccharide is selected from maltotriose and raffinose; a saccharide linked to an amino acid is D-ribose-L-cysteine; a saccharide linked with a polyphenol is selected from (-)-epigallocatechin gallate 3′-O-α-D-glucoside, isoquercitrin, baicalin and puerarin; and a saccharide linked with a lipid is a cerebroside, such as glucocerebroside, optionally wherein the active agent is bound to the carbohydrate via a linker.

5-6. (canceled)

7. The isolated EVs according to claim 1, wherein the active agent is a nucleic acid.

8. The isolated EVs according to claim 7, wherein the nucleic acid is an oligonucleotide, optionally wherein the oligonucleotide is selected from RNA, RNAi, siRNA, shRNA, miRNA, and or short activating RNA (saRNA).

9-10. (canceled)

11. The isolated EVs cargo according claim 1, wherein the active agent is covalently bound to a carbohydrate or derivative thereof via a cleavable linkage.

12. A method of loading isolated extracellular vesicles (EVs) with cargo molecules, comprising incubating a population of EVs with the carbohydrate molecules as an active agent or with cargo molecules comprising an active agent chemically bound to at least one carbohydrate or derivative thereof, wherein the active agent is selected from a small molecule, protein, peptide, lipid, polypeptide, and a nucleic acid.

13. The method according to claim 12, wherein the carbohydrate is selected from monosaccharide, disaccharide, trisaccharide, tetrasaccharide and oligosaccharide and wherein the carbohydrate derivative is selected from an amino acid, polyphenol, and lipid linked to a carbohydrate, optionally wherein the active agent is bound to said carbohydrate via a linker.

14. The method according to claim 13, wherein:
(i) the monosaccharide is selected from glucose, ribose, arabinose, galactose, mannose, and xylose,
(ii) the disaccharide is selected from sucrose, lactose and maltose;
(iii) the trisaccharide is maltotriose; or
(iv) the derivative comprising a saccharide linked with an amino acid is from D-ribose-L-cysteine;
v) derivative comprising a saccharide linked with a polyphenol is selected from (-)-epigallocatechin gallate 3′-O-α-D-glucoside, isoquercitrin, baicalin, puerarin, flavonoids, and isoflavonoids;
vi) the derivative comprising a saccharide linked with a lipid is cerebroside, optionally wherein the cerebroside is glucocerebroside.

15-21. (canceled)

22. The method according to claim 12, characterized by at least one of:
(i) the linker is DBCO-C6-acid;
(ii) the active agent molecule is a nucleic acid;
(iii) the active agent molecule is a nucleic acid is an oligonucleotide, optionally wherein the oligonucleotide is selected from RNA, RNAi, siRNA, shRNA, saRNA, and miRNA inhibitors;
(iv) the method is performed in the presence of insulin, optionally wherein the amount of the loaded exogenous cargo molecule in the resulting EVs is at least 20% higher than in EVs loaded in the absence of insulin;
(v) the EVs are exosomes; and
(vi) the extracellular vesicles are derived from adherent cells expressing mesenchymal markers. optionally wherein the adherent cells expressing mesenchymal markers are mesenchymal stem cells (MSC).

23-27. (canceled)

28. The method according to claim 12, wherein the active agent molecule is a nucleic acid bound to a carbohydrate selected from glucose and sucrose.

29. The method according to claim 12, wherein the method comprising electroporation or the use of a transfection reagent such as a lipid transfection reagent or wherein the method takes place in the absence of electroporation and in the absence of a transfection reagent.

30-36. (canceled)

37. Isolated EVs obtainable or obtained by the method of claim 12.

38. A pharmaceutical composition comprising a population of the isolated EVs comprising an exogenous cargo molecule comprising an active agent according to claim 1, and a pharmaceutically acceptable carrier and/or excipient.

39-41. (canceled)

42. An exogenous conjugate molecule comprising a nucleic acid covalently bound to at least one carbohydrate or derivative thereof.

43. The conjugate molecule according to claim 42, wherein the carbohydrate is selected from a monosaccharide, disaccharide, trisaccharide, tetrasaccharide and oligosaccharide and wherein the carbohydrate derivative is selected from a saccharide linked to an amino acid, polyphenol, or lipid, optionally wherein the monosaccharide is selected from glucose, ribose, arabinose, galactose, mannose, and xylose; the disaccharide is selected from sucrose lactose and maltose; the trisaccharide is selected from maltotriose and lactose; a saccharide linked to an amino acid is D-ribose-L-cysteine; a saccharide linked with a polyphenol is selected from (-)-epigallocatechin gallate 3′-O-α-D-glucoside, isoquercitrin, baicalin and puerarin; and a saccharide linked with a lipid is a cerebroside, such as glucocerebroside.

44. (canceled)

45. The conjugate molecule according to claim 42 to 44, wherein at least one of;
(i) the nucleic acid is bound to a carbohydrate via a linker; and
(ii) the nucleic acid is an oligonucleotide.

46. (canceled)

47. The conjugate molecule according to claim 46, wherein the oligonucleotide is selected from RNA, RNAi, siRNA, shRNA, miRNA, and saRNA.

48. The conjugate molecule according to claim 47, wherein the oligonucleotide is siRNA.

49. The conjugate molecule according to claim 48, wherein the conjugate comprises siRNA bound to a carbohydrate selected from glucose and sucrose.

50. (canceled)
